(19) United States
(12) Patent Application Publication
(10) Pub. No.: US 2011/0052644 A1
(43) Pub. Date: Mar. 3, 2011

(54) COMPOSITION COMPRISING EXTRACT OF CINNAMOMUM CASSIA BARK FOR IMPROVING NORMAL FLORA AND ENHANCING IMMUNE RESPONSE

(75) Inventors: Byoung Seob KO, Cheonan-si (KR); Won Kyung JEON, Seoul (KR); Sin-Hyeog IM, Gwangju (KR); Ho-Keun KWON, Seoul (KR); Ji-Sun HWANG, Gyeongju-si (KR); Dae Young KWON, Seongnam-si (KR)

(73) Assignees: KOREA INSTITUTE OF ORIENTAL MEDICINE, Daejeon (KR); KOREA FOOD RESEARCH INSTITUTE, Seongnam-si (KR)

(21) Appl. No.: 12/914,613

(22) Filed: Oct. 28, 2010

Related U.S. Application Data
(63) Continuation of application No. 11/916,189, filed on Nov. 30, 2007, now abandoned.

(30) Foreign Application Priority Data
Oct. 26, 2005 (KR) 10-2005-0101034
Oct. 25, 2006 (KR) PCT/KR2006/004381

(51) Int. Cl. A61K 36/54; A61P 37/04; A61P 1/00

(52) U.S. Cl. 424/278.1; 424/739

(57) ABSTRACT

The present invention relates to a composition for improving intestinal flora and enhancing immune response containing Cinnamomum cassia bark extract as an effective ingredient, more precisely, a composition containing Cinnamomum cassia bark extract is an effective ingredient that has the effects of increasing the growth of such intestinal beneficial bacteria as Bifidobacterium longum, Lacotobacillus sp. and Lactobacillus acidophilus and enhancing the proliferation of immune cells such as lymphocytes of general immune system as well as increasing the activity of intestinal immune cells. The composition of the present invention can be effectively used as a therapeutic agent for constipation or other intestine-related diseases and an immune enhancer owing to its activity of increasing immunity, particularly intestinal immunity, by increasing immune cell proliferation.

### CROSS REFERENCE TO RELATED APPLICATIONS

This application is a continuation of U.S. patent application Ser. No. 11/916,189 filed on Nov. 30, 2007, which is a 371 of PCT/KR2006/004381 filed on Oct. 25, 2006, which claims the benefit of Korean Application No. 10-2005-0101034 filed on Oct. 26, 2005, the contents of each of which are incorporated herein by reference.

### TECHNICAL FIELD

The present invention relates to a composition comprising the extract of Cinnamomum cassia bark as an effective ingredient for improving normal flora and enhancing immune response, more precisely a pharmaceutical composition containing the extract of Cinnamomum cassia bark which has functions of increasing the growth of Bifidobacterium longum, Lacotobacillus sp. and Lactobacillus acidophilus, increasing the proliferation of immune cells such as general lymphocytes, and activating immune cells of the intestinal immune system.

### BACKGROUND ART

From the birth, human system becomes a habitat for intestinal microorganisms, and once the microorganisms are in equilibrium, they form a stable intestinal bacterial flora. The intestinal bacterial flora is affected by the conditions of a host including physiological condition, food, medicine, stress, etc.

The numbers and activity of microorganisms forming the normal flora are regulated by an allogenic factor and an autogenic factor (Yazawa T., Letters in applied Microbiology 10: 229-232, 1990). The allogenic factor is derived from the environment surrounding a host and its diet, while the autogenic factor is generated among intrafloral microorganisms.

The balance among the normal flora is accomplished by the competition for a habitat and nutrition under the conditions of strong anaerobic condition, peristaltic movement of intestines, and continuous excretion, etc. It was additionally reported that the balance of the normal flora is also regulated by factors such as pH, oxidation-reduction potential, bile acid, bacteriocin, fatty acid, and hydrogen sulfide (Yazawa K. and Tamura Z. Bifidobacteria Microflora 1(1): 39-44, 1982).

Each microorganism composing the intestinal flora is beneficial or harmful for a host depending on its ability of production or decomposition. For example, the intestinal flora, as a whole, is involved in providing vitamin, preventing infection and helping the original functions of intestines (peristaltic movement and absorption). Therefore, the composition of the flora is closely related to constipation and other intestine-related diseases (Mistuoka T. Bifidobacteria Microflora, 1 (1): 3, 1982). In aged people and those having weak intestines, the abnormal intestinal flora is observed (Mitsuoka T, Journal of Industrial Microbiology, 6:263, 1990). In general, the population of the flora is significantly increased in small intestines. Particularly, Bifidobacterium ssp (benefit bacteria) is reduced or extinguished, whereas Clostridium ssp such as C. perfringers is significantly increased. So, it is important for long healthy life that the intestinal flora is as balanced as possible by lowering harmful microorganisms such as C. perfringers and increasing helpful microorganisms such as Bifidobacterium ssp (Mitusoka T., Ecology and role of intestinal flora., Japan Scientific Society Press, Tokyo, p. 1, 1989).

Cinnamomum cassia bark is the outer skin of an evergreen tall tree belonging to the Lauraceae, which is distributed in southern area of China and Vietnam. Cinnamomum cassia bark indicates the outer skin, Cinnamomum cassia stem indicates the branch, indicates the thick bark, and indicates the dried old and thick outer skin of the tree. Cinnamomum cassia bark contains 1-3.4% of essential oil (cinnamic aldehyde 75-90%, cinnamyl aldehyde, etc), 2-3% of tannin, mucus and carbohydrates, and the higher essential oil content is observed in the bark of the 5-6 year old tree. The Cinnamomum cassia bark has long been used as a diaphoretic, a febrifuge, an anodyne and as a spice. It has been reported that the Cinnamomum cassia bark has functions of enhancing peristaltic movement but inhibiting abnormal fermentation in intestines. However, the mechanism of such actions has not been explained, yet.

The Cinnamomum cassia bark has also been used as an Oriental folk medicine prescribed for those having weak constitutions and weak Qi-Blood (energy and blood) in order to improve immune response of those. However, the precise mechanism to aid immune response has not been understood, either. Therefore, it was the present inventors' guess that observing the effect of the Cinnamomum cassia bark extract on leukocytes (lymphocytes, plasma cells, phagocytes, and granulocytes) and lymphatic organ involved in immune response may lead to the explanation of exact mechanism of the Cinnamomum cassia bark in relation to the enhancement of immune response. In particular, intestinal immune system is a good example for immunological tolerance induced where there are various food antigens and normal floras together. Precisely, intestinal immune system does not respond to harmless antigens such as food or normal flora, while the system selectively responds to harmful antigens such as virus or pathogenic bacteria (immunological homeostasis). The immunological homeostasis in the intestines is maintained normally by immunological tolerance in oral cavity for harmless antigens and immune response against harmful antigens. However, once immunological tolerance is frustrated by any intestinal mechanism, immune system responds to antigens included in food or normal flora and even intestinal wall itself, causing inflammatory enteritis which brings disorders of digestive function and excretion function of the intestines. The intestinal immunity is found in intestine-related lymphoreticular tissue, which is one of three mucous lymphatic organs, taking at least ⅓ of in vivo lymphatic tissue and belonging to the second lymphatic tissue of the two lymphatic tissues, and is observed on mucous membrane of the intestines playing an important role in self-defense by inducing IgA response in the intestines, etc (Bienestock, J. et. al., Immunol., 41, 249-270 (1980)). The key action of the intestinal immunity is to activate cells by eating a soluble antigen, virus and bacteria by pinocytosis or phagocytosis and then migrate them to lymphocytes (Trier, J., Gastroenterol. Clin. North Am., 20, 531-547 (1991)).

There are some patents describe a composition containing Cinnamomum cassia bark extract, for example a preventive and therapeutic composition for arteriosclerosis containing the extract of Cinnamomum cassia bark (Korean Patent No. 10-1998-0021474), a composition for cosmetics containing the extract of Cinnamomum cassia bark (Korean Patent No. 10-1999-0034707) and a composition for improving dental hygiene containing the nano-sized extract of Cinnamomum cassia bark (Korean Patent No. 10-2002-0074210). However, there have been no descriptions on Cinnamomum cassia bark in relation to the activities of improving intestinal flora and enhancing immune response.

Thus, the present inventors studied the extract of Cinnamomum cassia bark and confirmed that a composition containing the extract of Cinnamomum cassia bark as an effective ingredient increases the populations of Bifidobacterium longum, Lacotobacillus sp. and Lactobacillus acidophilus, which are all beneficial bacteria, and helps the proliferation of immune cells such as lymphocytes in the spleen and the intestines, and thereby the inventors further completed this invention by confirming that the composition of the invention can be used as a therapeutic agent for constipation and other intestine-related diseases and an immune enhancer, particularly intestinal immune enhancer, based on our findings.

### DISCLOSURE

### Technical Problem

It is an object of the present invention to provide a composition containing the extract of Cinnamomum cassia bark as an effective ingredient for improving intestinal flora.

It is also an object of the present invention to provide a composition containing the extract of Cinnamomum cassia bark as an effective ingredient for enhancing immune response.

It is further an object of the present invention to provide a composition containing the extract of Cinnamomum cassia bark as an effective ingredient for enhancing intestinal immunity.

It is also an object of the present invention to provide a health food containing the extract of Cinnamomum cassia bark for enhancing immune response.

### Technical Solution

To achieve the above objects, the present invention provides a composition containing the extract of Cinnamomum cassia bark for improving intestinal flora.

The present invention also provides a composition containing the extract of Cinnamomum cassia bark for enhancing immune response.

The present invention further provides a composition containing the extract of Cinnamomum cassia bark for enhancing intestinal immunity.

The present invention also provides a method for improving intestinal flora which includes the step of administrating the effective dose of the extract of Cinnamomum cassia bark for improving intestinal flora.

The present invention also provides a method for enhancing immunity which includes the step of administrating the effective dose of the extract of Cinnamomum cassia bark to a patient who is in need of immunity enhancement.

The present invention also provides a method for treating intestine-related diseases which includes the step of administrating the pharmaceutically effective dose of the extract of Cinnamomum cassia bark to a patient in need.

The present invention also provides health foods for enhancing immunity containing the extract of Cinnamomum cassia bark as an effective ingredient.

The present invention also provides a method for improving intestinal flora in a subject comprising administering an effective amount of the extract of Cinnamomum cassia bark to the subject in need thereof.

The present invention also provides a method for enhancing immune response in a subject comprising administering an effective amount of the extract of Cinnamomum cassia bark to the subject in need thereof.

The present invention also provides a method for treating constipation or intestine-related diseases in a subject comprising administering an effective amount of the extract of Cinnamomum cassia bark to the subject in need thereof.

Hereinafter, the present invention is described in detail.

The present invention provides a composition containing the extract of Cinnamomum cassia bark for improving intestinal flora.

The present invention also provides a method for improving intestinal flora in a subject comprising administering an effective amount of the extract of Cinnamomum cassia bark to the subject in need thereof.

The present invention also provides a method for treating constipation or intestine-related diseases in a subject comprising administering an effective amount of the extract of Cinnamomum cassia bark to the subject in need thereof.

Cinnamomum cassia bark is the outer skin of an evergreen tall tree belonging to the Lauraceae, which is distributed in southern area of China and Vietnam. The Cinnamomum cassia bark has long been used as a diaphoretic, a febrifuge, an anodyne and as a spice. It has been reported that the Cinnamomum cassia bark has functions of enhancing peristaltic movement but inhibiting abnormal fermentation in intestines. However, the mechanism of such actions has not been explained, yet.

Microorganisms residing in the intestines begin to growth and form an intestinal flora once the population is in equilibrium. Each microorganism compositing the intestinal flora is involved in vitamin supply, prevention of infection and other functions of the intestines, suggesting that those microorganisms are closely related to constipation and other intestine-related diseases (Mistuoka T. Bifidobacteria Microflora, 1(1): 3, 1982).

The present inventors investigated the activity of the extract of Cinnamomum cassia bark, known to have the intestine function enhancing effect, to intestinal beneficial bacteria. First of all, dried Cinnamomum cassia bark was obtained (Hwajin Distribution Co.), followed by extraction for 3 hours in a hot water extractor. The extract was filtered with a filter paper, concentrated with a vacuum evaporator and freeze-dried to give powders. The powder extract was suspended in sterilized distilled water at a proper concentration and filtered whenever it was needed for an experiment.

The extract of Cinnamomum cassia bark of the present invention is extracted by using one or more solvents selected from a group consisting of water, single or mixed ether, ethanol, methanol and ethyl acetate, and then concentrated under reduced pressure. The solvent herein is preferably water and the extraction method can be one of hot water extraction, maceration, reflux or ultrasonic extraction, but hot water extraction is preferred.

Among various intestinal floras, Bifidobacterium longum (ATCC 15707), Lactobacillus sp. (KCTC 3930), and Lactobacillus acidophilus (ATCC 4356) are used as an index for intestinal beneficial bacteria. Those strains were cultured in Reinforced Clostridial Media (RCM) in a 37° C. BBL GasPak (Becton Dickinson and Company) under anaerobic condition.

The present inventors investigated the effect of the extract of Cinnamomum cassia bark on the growth of Bifidobacterium longum, Lactobacillus sp., and Lactobacillus acidophilus strains. For the experiment, the powder extract was added to media with different concentrations, to which Bifidobacterium longum, Lactobacillus sp., and Lactobacillus acidophilus pre-culture solutions were inoculated. O.D (optical density) was measured to investigate the growth of those strains. As the amount of the extract of Cinnamomum cassia bark was increased, the growth of the strains was increased, O.D. was higher and growth activation ratio (OD of experimental group/OD of control group) was increased (see FIG. 1).

Different concentrations of the extract of Cinnamomum cassia bark were loaded on 6 mm disc paper, followed by applying on a solid medium. After culturing the strains, the size of a growth activity zone was measured. As shown in Table 1, Bifidobacterium longum, Lactobacillus sp., and Lactobacillus acidophilus strains cultured on the solid medium loaded with 10 mg/disc of the extract of Cinnamomum cassia bark exhibited big growth activation zones, indicating that the extract of Cinnamomum cassia bark of the present invention has a function of promoting the growth of intestinal beneficial bacteria (see Table 1).

The extract of Cinnamomum cassia bark was orally administered to a mouse and the blood composition of the mouse was investigated. Particularly, 500 mg/kg/day of the extract of Cinnamomum cassia bark was orally administered to a mouse for 15 days, followed by observation. As a result, the blood composition of the mouse was not very different from that of a control (see Table 2).

The present invention also provides a composition containing the extract of Cinnamomum cassia bark for enhancing immune response.

The present invention also provides a method for enhancing immune response in a subject comprising administering an effective amount of the extract of Cinnamomum cassia bark to the subject in need thereof.

The present inventors further investigated if the extract of Cinnamomum cassia bark, which has been used as an Oriental folk medicine for supplement of Gi-Hyul (energy and blood), had the function of enhancing immune cell proliferation in addition to the already confirmed function of promoting the growth of intestinal beneficial bacteria. For the investigation, T cells were separated from the spleen of a mouse administered with the extract of Cinnamomum cassia bark and cell division capacity thereof was investigated. As a result, high cell division capacity was confirmed (FIG. 9).

The present invention also provides a composition containing the extract of Cinnamomum cassia bark for enhancing intestinal immunity. The intestinal immunity has a big difference in its mechanism and operating area with the general humoral immunity. So, even a substance has the function of promoting humoral immunity, this substance does not necessarily have the function of promoting intestinal immunity as well. To investigate the effect of the extract of Cinnamomum cassia bark on the intestinal immunity and general immune system, the present inventors administered the extract orally to a mouse and measured the cytokine expressions of intestinal immune cells (mesenteric lymph node T cells and B cells, lamina propria mononuclear cells) which are indexes for intestinal immune response. As a result, when the extract was administered into the intestines, it can obviously regulate the intestinal immune activity by regulating the cytokine expression which is involved in the increase and decrease of immunity (see FIG. 2-FIG. 8 and FIGS. 10-13).

The pharmaceutical composition for improving intestinal flora and enhancing immune response of the present invention contains the extract of Cinnamomum cassia bark as an effective ingredient. The extract of Cinnamomum cassia bark can be administered orally or parenterally and be used in general forms of pharmaceutical formulation. Solid formulations for oral administration are tablets, hard or soft capsules, solutions and suspensions. Those pharmaceutical formulations can be prepared by mixing the extract with generally used fillers, extenders, binders, wetting agents, disintegrating agents, diluents such as surfactant, or excipients.

The effective dosage of the extract of Cinnamomum cassia bark can be determined according to weight, age, gender, health condition, diet, administration frequency, administration method, excretion and severity of a disease. The dosage of the extract of Cinnamomum cassia bark is 0.1 mg-10 g/kg per day, and preferably 10 mg-1 g/kg per day. An individual dose preferably contains the effective amount of the active compound which can be administered in one application and which usually corresponds to a whole, ½, ⅓ or ¼ of a daily dose. Administration frequency is 1-6 a day. However, the content of the extract might be less than the above when it is administered for long-term to improve health conditions but the effective dosage could contain more than the above amount because the extract of the invention is very safe.

The Cinnamomum cassia bark, a raw material of the extract of the invention was proved to be a safe substance. The Cinnamomum cassia bark was orally administered to rats to investigate toxicity. As a result, it was evaluated to be safe substance since its estimated LD₅₀ value is much greater than 10 g/kg in rats.

The present invention also provides health foods for improving intestinal flora and enhancing immune response containing the extract of Cinnamomum cassia bark as an effective ingredient.

The extract of Cinnamomum cassia bark of the present invention can be included in health food. At this time, the extract of Cinnamomum cassia bark can be added as it is or after being mixed with other food or ingredients, according to the conventional method. The mixing ratio of effective ingredients is determined by the purpose of use (prevention, health or therapeutic treatment). In the case of producing food or beverages containing the extract of Cinnamomum cassia bark of the present invention, the extract is preferably added by 40-70 weight %, more preferably 50-60 weight %, to the raw material. However, the content of the extract might be less than the above when it is administered for long-term to improve health conditions but the effective dosage could contain more than the above amount because the extract of the invention is very safe.

There is no limit in applicable food, which is exemplified by meats, sausages, bread, chocolate, candies, snacks, cookies, pizza, ramyun, noodles, dairy products including ice cream, soups, beverages, tea, drinks, alcoholic drinks and vitamin complex, etc, and in fact every health food generally produced are all included.

### DESCRIPTION OF DRAWINGS

The application of the preferred embodiments of the present invention is best understood with reference to the accompanying drawings, wherein:

FIG. 1 is a graph illustrating the growth activity of intestinal beneficial bacteria in a liquid medium containing the extract of Cinnamomum cassia bark.

FIG. 2 is a graph illustrating the level of TNF-α expression by T cells separated from a mouse mesenteric lymph node according to the oral-administration of the extract of Cinnamomum cassia bark, and

FIG. 3 is a graph illustrating the level of IFN-γ expression by T cells separated from a mouse mesenteric lymph node according to the oral-administration of the extract of Cinnamomum cassia bark.

FIG. 4-FIG. 7 are graphs illustrating the expression levels of cytokines induced by B cells separated from a mouse mesenteric lymph node according to the oral-administration of the extract of Cinnamomum cassia bark, FIGS. 4, 5, 6 and 7 illustrate the expression levels of IL-4, IL-10, IFN-γ and INF-α, respectively.

FIG. 8 is a graph illustrating the level of IFN-γ expression by mononuclear cells separated from a mouse lamina propria according to the oral-administration of the extract of Cinnamomum cassia bark.

FIG. 9 is a graph illustrating T-cell proliferation stimulated by anti-CD3 antibody and anti-CD28 antibody in CD⁴⁺ T-cells separated from the spleen and mesenteric lymph node according to the oral-administration of the extract of Cinnamomum cassia bark.

FIG. 10 is a graph illustrating the results of investigation on the intracellular levels of TNF-α by using a flow cytometer. CD⁴⁺ T-cells were separated from the spleen and mesenteric lymph node after the oral-administration of the extract of Cinnamomum cassia bark and then those cells were stimulated by PMA and ionomycin for 6 hours, followed by measuring the levels of INF-α with flow cytometry.

FIG. 11 is a graph illustrating the results of investigation on the intracellular levels of INF-α by using a flow cytometer. B-cells were separated from the spleen and mesenteric lymph node after the oral-administration of the extract of Cinnamomum cassia bark and then those cells were stimulated by PMA and ionomycin for 6 hours, followed by measuring the levels of INF-α with flow cytometry.

FIG. 12 is a graph illustrating the results of investigation on the intracellular levels of IFN-γ by using a flow cytometer. CD⁴⁺ T-cells were separated from the spleen and mesenteric lymph node after the oral-administration of the extract of Cinnamomum cassia bark and then those cells were stimulated by PMA and ionomycin for 6 hours, followed by measuring the levels of IFN-γ with flow cytometry.

FIG. 13 is a graph illustrating the results of investigation on the intracellular levels of IFN-γ by using a flow cytometer. B-cells were separated from the spleen and mesenteric lymph node after the oral-administration of the extract of Cinnamomum cassia bark and then those cells were stimulated by PMA and ionomycin for 6 hours, followed by measuring the levels of IFN-γ with flow cytometry.

FIG. 14 is a set of graphs illustrating that the Cinnamomum cassia bark extract dependent expression level of a reporter gene in mouse lymphoma T-cells, EL4 cells, expressed by TNF-α reporter vector and the expression level of a reporter gene according to the co-treatment of PMA and ionomycin with the Cinnamomum cassia bark extract.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Materials

### <1-1> Preparation of the Extract of Cinnamomum Cassia Bark

The dried Cinnamomum cassia bark purchased from Hwajin Distribution Co. was pulverized, followed by hot water extraction for three hours in a hot water extractor. The Cinnamomum cassia bark hot water extract was filtered with a filter paper and the supernatant was concentrated with a rotary evaporator. The extract was then freeze-dried by using a freeze-dryer (Ilshin) to give a powder extract. The powder extract was suspended in sterilized distilled water to prepare proper concentrations, which were used after being filtered.

### <1-2> Preparation of Enterobacteria

Bifidobacterium longum (ATCC 15707), Lactobacillus sp. (KCTC 3930), and Lactobacillus acidophilus (ATCC 4356) were used as indexes for intestinal beneficial bacteria. Those strains were cultured in Reinforced Clostridial Media (RCM, Difco, USA) in a 37° C. BBL GasPak (Becton Dickinson and Company, USA) under anaerobic condition.

### Example 2

### Effect of the Extract of Cinnamomum Cassia Bark on the Growth of the Intestinal Beneficial Bacteria

To investigate the effect of the extract of Cinnamomum cassia bark on the growth of the intestinal beneficial bacteria (Bifidobacterium longum, Lactobacillus sp. and Lactobacillus acidophilus strains), experiments were performed as follows. Each experiment was repeated three times and the result was presented by the ratio of OD value of an experimental group treated with the extract to a control group treated with water only.

### <2-1> Growth Activity Test by Suspension Culture

To investigate the effect of the extract of Cinnamomum cassia bark on the growth of intestinal beneficial bacteria, a modified EG (Eggerth Gagnon) medium (beef extract 2 g, proteose peptone No. 3 10 g, yeast extract 5 g, Na₂HPO₄ 4 g, soluble starch 0.5 g, glucose 1.5 g, L-cysteine 0.4 g, silicon antifoamer 0.25 ml, Tween 80 0.5 g, D.W 1,000 ml) was prepared with the addition of the freeze-dried Cinnamomum cassia bark extract at the concentrations of 100 ug, 1 mg, and 10 mg/ml. To the medium, the pre-culture solutions of Bifidobacterium longum, Lactobacillus sp. and Lactobacillus acidophilus activated under anaerobic condition were inoculated, followed by further culture for 18 hours at 37° C. under anaerobic condition. OD₆₀₀ was measured to judge the growth of those strains.

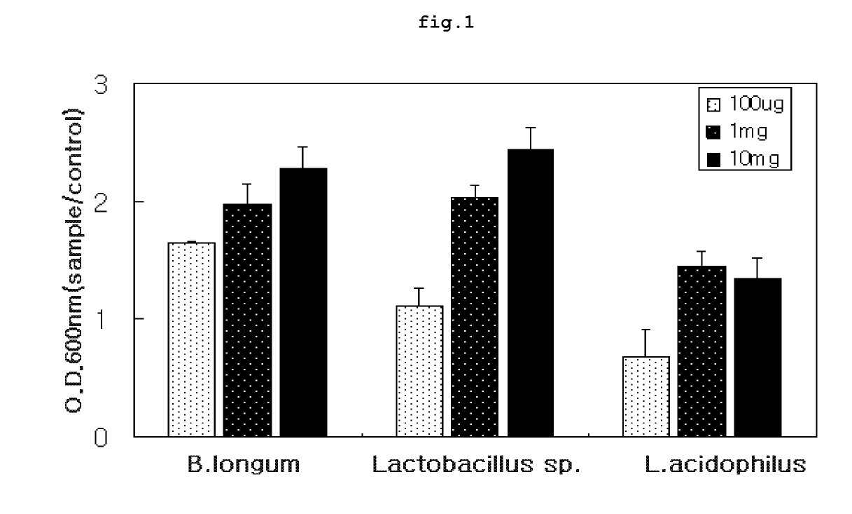

The results are presented by the ratio of OD of the experimental group treated with the extract to OD of the control group treated with water only. From the results of measuring the growth of intestinal beneficial bacteria in the liquid medium containing the extract of Cinnamomum cassia bark was confirmed, as shown in FIG. 1, that the growths of the strains were enhanced with the increase of the extract of Cinnamomum cassia bark with exhibiting the increase of OD. For example, when 10 mg/ml of the extract of Cinnamomum cassia bark was added, the growth of Lactobacillus sp. was 2.4-fold higher, compared with that of a control. And, the growths of Bifidobacterium longum and Lactobacillus acidophilus strains were also enhanced with the increase of the extract of Cinnamomum cassia bark.

### <2-2> Growth Activity Test by Agar Diffusion Method

After sterilizing, EG agar medium was cooled at 50° C. and divided into three plates each containing 2-3% of each strain. The medium was hardened at room temperature. The extract of Cinnamomum cassia bark was loaded on 6 mm disc papers (Whatman paper No. 41) at different concentrations of 0.1 mg, 1 mg, and 10 mg, which were distributed at regular intervals on the solid medium. The medium was cultured at 37° C. for 48 hours under anaerobic condition and the size of a growth activity ring was measured. As shown in Table 1, in the solid medium containing 10 mg/disc of the extract of Cinnamomum cassia bark, Bifidobacterium longum exhibited + growth activity ring and so did Lactobacillus sp., while Lactobacillus acidophilus exhibited +++ growth activity ring. The results indicate that the extract of Cinnamomum cassia bark has an excellent growth enhancing activity.

Table 1: The activity to improve intestinal flora of the extract of Cinnamomum cassia bark

TABLE 1

| | Dose (mg/disc) | | |
|---|---|---|---|
| Strain | 0.1 | 1 | 10 |
| B. longum | n | n | + |
| C. perfringens | n | − | − |
| Lactobacillus sp. | n | + | + |
| L. acidophilus | + | ++ | +++ |

n: no effect, +: 10-14 mm in diameter, ++: 15-19 mm in diameter, +++: 20-24 mm in diameter, −: inhibitory zone, 10-14 mm in diameter

### Example 3

### The Effect of the Extract of Cinnamomum Cassia Bark on Blood Composition According to the Oral-Administration to a Mouse

The present inventors orally administered the extract of Cinnamomum cassia bark to a mouse and investigated the blood composition. Six-week old male mice were divided into two groups: a control group and an experimental group (3 mice/group). And the experimental group was orally administered with the extract of Cinnamomum cassia bark by 500 mg/kg/day for days. The blood composition was investigated by using a coulter counter (COLUTER JT) and the results are shown in Table 2.

TABLE 2

| | | Control | Experimental |
|---|---|---|---|
| Test | Definition | group | group |
| Weight | Weight | 38.49 ± 2.356b) | 36.61 ± 3.192 |
| WBC | Leukocyte | 2.77 ± 0.831 | 2.5 ± 1.127 |
| LY | Lymphocyte | 71.77 ± 6.725 | 53 ± 9.582 |
| MO | Mononuclear cell | 9.83 ± 3.85 | 6.4 ± 4.667 |
| GR | Granulocytes | 18.40 ± 5.449 | 38.73 ± 13.462 |
| LY# | Lymphocyte | 2.00 ± 0.648 | 1.3 ± 0.656 |
| MO# | Mononuclear cell | 0.28 ± 0.117 | 0.2 ± 0.141 |
| GR# | Granulocyte | 0.48 ± 0.223 | 1 ± 0.656 |
| RBC | Erythrocyte | 6.18 ± 0.939 | 6.34 ± 0.333 |
| HGB | Hemoglobin | 10.13 ± 1.568 | 10.33 ± 0.416 |
| HCT | Hematocrit | 31.57 ± 4.807 | 33.13 ± 1.115 |
| MCV | Mean corpuscular | 51.11 ± 1.426 | 52.3 ± 1.646 |
| | volume | | |
| MCH | Mean corpuscular | 16.39 ± 0.306 | 16.3 ± 0.529 |
| | hemoglobin | | |
| MCHC | Mean corpuscular | 32.06 ± 0.712 | 31.17 ± 0.252 |
| | hemoglobin | | |
| | concentration | | |
| RDW | Erythrocyte | 16.54 ± 0.677 | 15.73 ± 1.415 |
| | distribution width | | |
| PLT | Thrombocyte | 88.00 ± 21.463 | 108.67 ± 16.773 |
| MPV | Mean thrombocyte | 4.24 ± 0.374 | 4.23 ± 0.153 |
| | volume | | |

As shown in Table 2, there was no significant difference in blood composition between the control and the extract treated experimental group.

### Example 4

### The Effect of the Extract of Cinnamomum Cassia Bark on the Mouse Intestinal Immunity

### <4-1> Separation of Mouse Intestinal Immune Cells

To investigate the effect of the extract of Cinnamomum cassia bark on the host immune system according to the oral-administration, mouse spleen, lymph node, mesenteric lymph node and lamina propria mononuclear cells were separated.

Particularly, after oral-administration of the extract of Cinnamomum cassia bark, mice were sacrificed and the spleen, mesenteric lymph node and intestines were extracted. T-cells and B-cells were isolated from the taken spleen and mesenteric lymph node by a magnetic method. The magnetic method indicates that T-cell and B-cell-specific magnetic beads are bound to T-cells and B-cells, which are placed on a strong magnetic field to fix the beads binding T-cells and B-cells on the magnetic field in order to eliminate non-specific cells and then the magnetic field is eliminated to obtain target T-cells and B-cells only.

To separate lamina propria mononuclear cells from the intestines, the intestines were first extracted and muscular tissues were removed by treating 1% collagenase (Type I collagenase, Sigma, USA) for three hours. Cells of the lamina propria layer under the muscular tissue were processed into single cells. Finally, the lamina propria mononuclear cells were obtained by fercoll density gradient.

### <4-2> Expression Level of Cytokine Secreted by T-Cells of the Mouse Mesenteric Lymph Node

Cells were stimulated by PMA (Sigma, USA) and ionomycin (Kalbiochem, Switzerland). To provide in vivo situation, CD3 and CD28 antibodies (BD bioscience, USA), which have been used for in vivo T cell assay, were used to stimulate the cells for 48 hours. Any changes in T-cells of the mesenteric lymph node were observed.

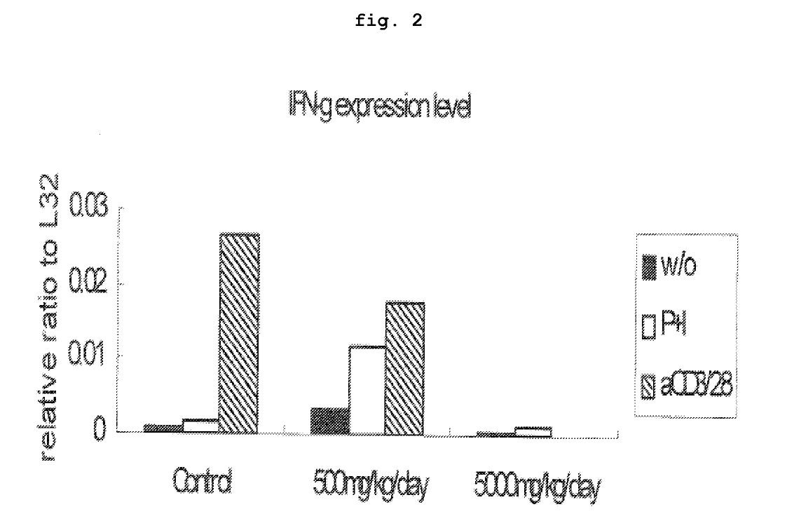

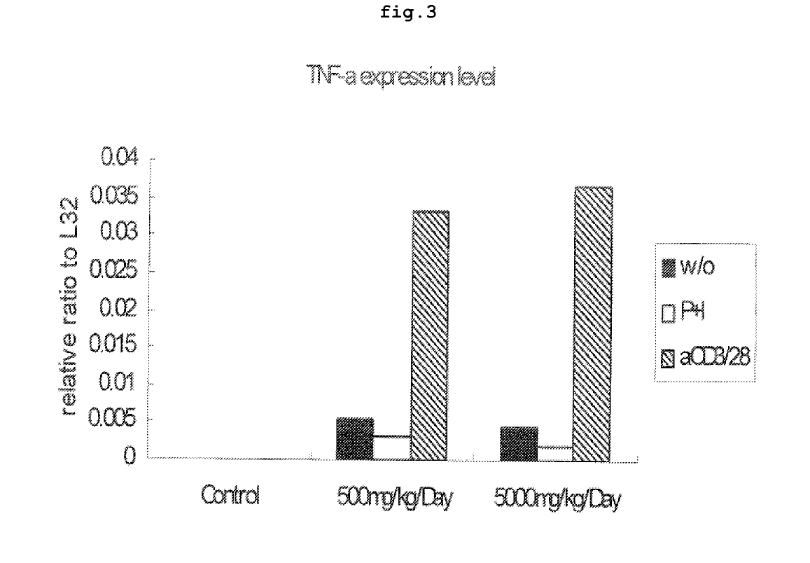

As a result, changes were observed in proteins (immune inducers) of the T-cells. First, the level of mRNA of TNF-α (tumor necrosis factor-alpha) was increased, compared with that in the control, regardless of stimulation, which was consistent with the increase of B-cells of mesenteric lymph node. However, expression pattern of interferon-gamma, which has been known to be induced by the extract of Cinnamomum cassia bark, was changed by the stimulation and in fact the expression was increased by the extract of Cinnamomum cassia bark (FIG. 2 and FIG. 3).

### <4-3> Expression Level of Cytokine Secreted by B-Cells of the Mouse Mesenteric Lymph Node

Mice were orally administered with the extract of Cinnamomum cassia bark for 10 days by 500 mg/kg/day and 5000 mg/kg/day. The mice were sacrificed and T-cells and B-cells were separated from the mesenteric lymph node by a magnetic method. The separated cells were stimulated by PMA and ionomycin and for in vivo like approach, LPS (lipopolyssacaride) that has been used to stimulate in vivo B-cells was used to stimulate B-cells for 4 hours. Then, the changes in B-cells of each group were observed.

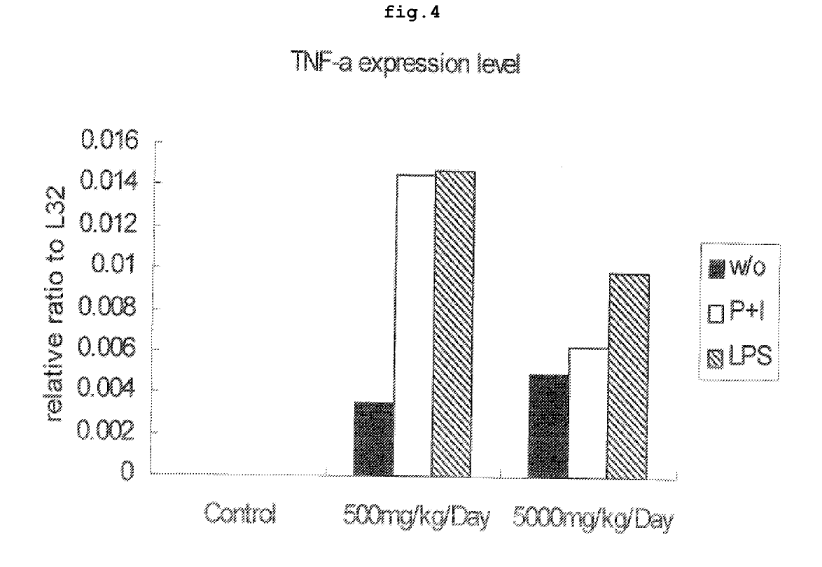

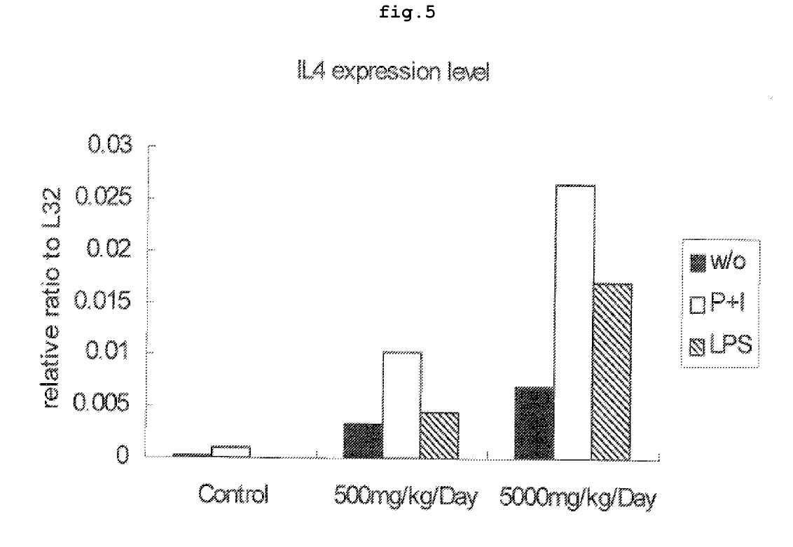

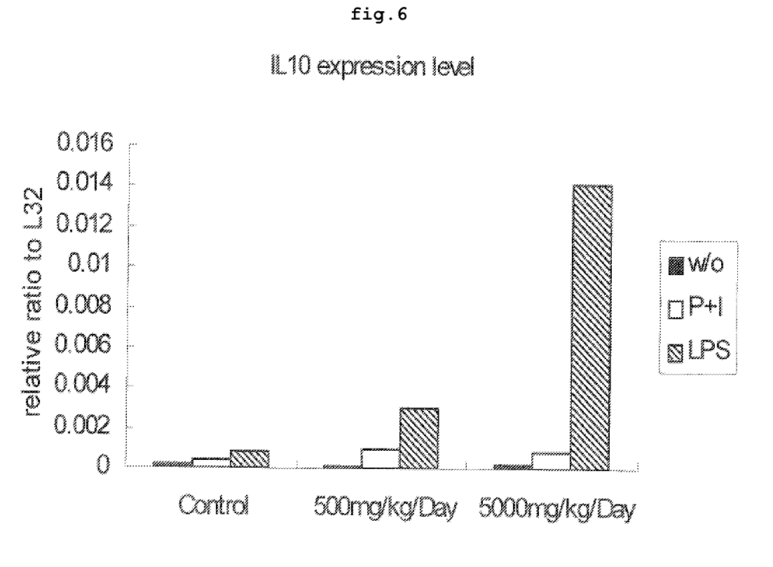

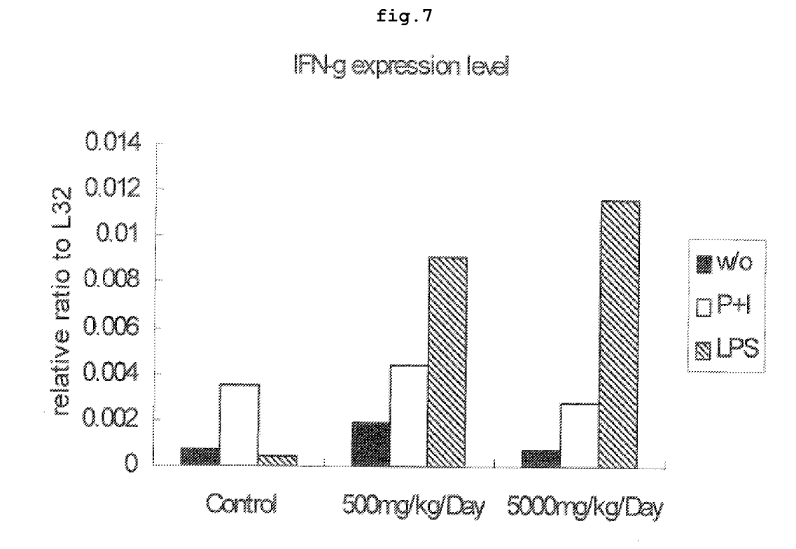

As a result, significant changes in molecules of the experimental group were observed. First, the expression level of TNF-α (tumor necrosis factor-alpha) was too low to detect in the control group, while over-expression of TNF-α was observed in both experimental groups treated by 500 mg/kg/day and 5000 mg/kg/day, regardless of the stimulation (FIG. 4). The expression pattern of interleukin-4 was also similar to that of TNF-α (FIG. 5). The expression level of interleukin-10 was slightly increased with or without stimulation with PMA/ionomycin, but significantly increased, approximately at least 100 fold increased compared with the control, with the stimulation with LPS (FIG. 6). The expression of interferon-gamma was also increased the extract dose dependently regardless of stimulation (FIG. 7).

### <4-4> Expression Level of Cytokine Secreted by Lamina Propria Mononuclear Cells

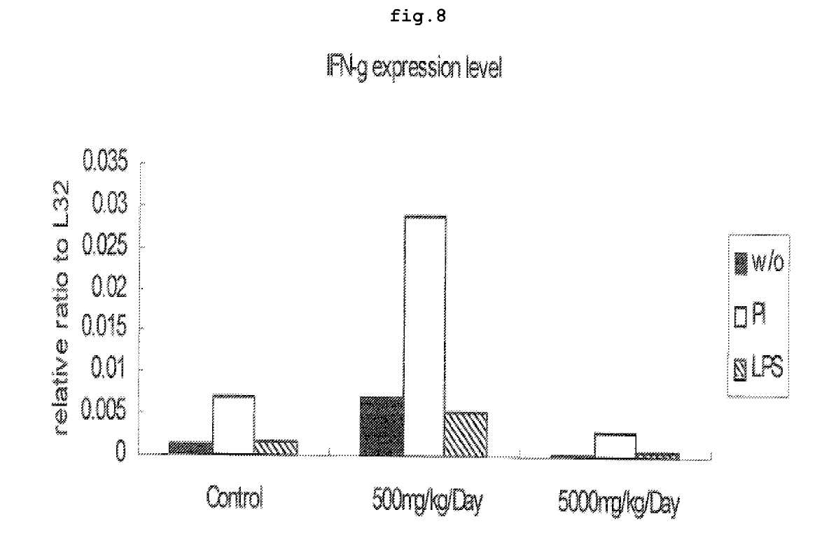

Lamina propria mononuclear cells are most important key cells for the study of the intestinal immune system. These immune cells are found under the intestinal epidermal cells and involved in regulating intestinal immunity. However, the cells are so difficult to obtain that many researchers have not been successful in using these cells for experiments. The present inventors previously established a method for obtaining the lamina propria mononuclear cells by the process described earlier. Therefore, the inventors could measure the Cinnamomum cassia bark extract dependent expression of mRNA of the cells with the same stimulation (PMA/Ionomycin, LPS and anti-CD3/anti-CD28 antibody) as the above. As a result, the expression of interferon-gamma was significantly increased by the administration of Cinnamomum cassia bark extract in lamina propria mononuclear cells, compared with that of the control (FIG. 8).

### Example 5

### The Effect of Cinnamomum Cassia Bark Extract on the Mouse Spleen and Mesenteric Lymph Node

<5-1> Separation of T-Helper Cells from the Mouse Spleen

To separate CD⁴⁺T helper cells from the mouse spleen, a mouse was sacrificed and the spleen was extracted. Then, the spleen tissues were physically crashed and flowed through a filter (40 μm nylon mesh; Falcon) to prepare single cells. Then, the CD⁴⁺ T cells were separated by a magnetic method and CD4-specific binding metal beads (Miltenyi Biotec, USA) were added to let the beads bound to the surface of CD⁴⁺ T cells. The cells were placed on a strong magnetic field to eliminate those cells not binding to beads. Then, the magnetic field was removed and the CD⁴⁺ T cells were obtained.

<5-2> The Effect of the Oral-Administration of Cinnamomum Cassia Bark Extract on Division Capacity of T cells of the Mouse Spleen and Mesenteric Lymph Node

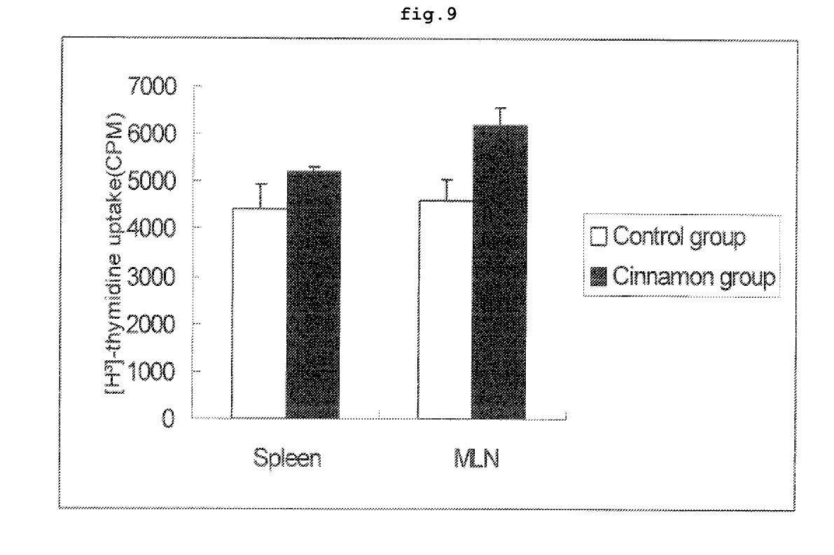

With reference to the Oriental folk medicine, the present inventors investigated the effect of the oral-administration of Cinnamomum cassia bark extract on division capacity of host T cells. Before the experiment, the Cinnamomum cassia bark extract had been oral-administered to mice for 8-20 weeks (500 mg/kg/1 time). Upon completion of the oral-administration, the mice were sacrificed to extract the spleen and mesenteric lymph node. CD⁴⁺ T helper cells were separated from the extracted tissues by the same manner as described above. To evaluate the division capacity of the separated T cells, equal amount of cells were stimulated with anti-CD3 antibody (1 ug/ml) and anti-CD28 antibody (1 ug/ml). The cells were cultured for three days after the stimulation to give enough time for cells to respond and be differentiated. The radio-isotope ([³H]-thymidine) was added and bound to intracellular DNA. The division capacity was evaluated by examining the increased level of cells by measuring the radioactivity of the radio-isotope therein. As a result, higher division capacity was observed in T cells treated with the Cinnamomum cassia bark extract than in T cells treated with PBS, in both the spleen and mesenteric lymph node (FIG. 9).

### <5-3> Expressions of Interferon-Gamma and INF-α in T Cells of the Mouse Spleen and Mesenteric Lymph Node According to the Oral-Administration of the Cinnamomum Cassia Bark Extract

Previous experiments confirmed that the oral-administration of Cinnamomum cassia bark extract increased the levels of mRNA of INF-γ and INF-α, the most representative cytokines involved in immunity, suggesting that the extract has an effect of enhancing immune response by T cells. Based on this result, the expressions of the above two cytokines were evaluated again at protein level in this embodiment. T cells and B cells were separated from the spleen and mesenteric lymph node of both experimental mice orally administered with Cinnamomum cassia bark extract and control mice treated with PBS only. To be able to secret cytokines, T cells and B cells separated above were stimulated by PMA and ionomycin for a while. To prevent the outward release of the secreted cytokines, the stimulated cells were treated with Brefeldin A (Epicentre biocompany; B901MG, USA), resulting in the prevention of migration of secreted proteins in ER. With the block of protein migration in ER, a hole was made on the cell membrane using permeabilization buffer (0.5% saponin, 1% BSA in D.W). Radio-labeled (anti-IFN-PE, anti-TNFa-PE) INF-γ and TNF-α were added to the cells having a hole on it, followed by measuring the levels of cytokines secreted in the cells by using a flow cytometer.

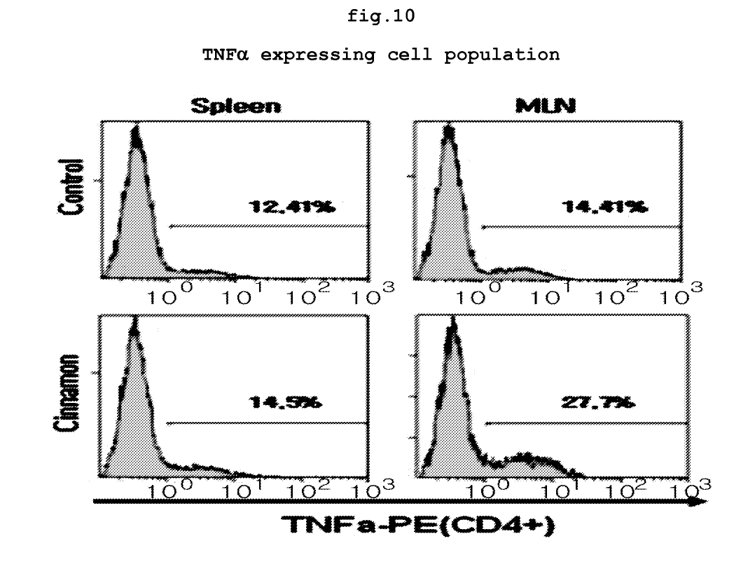

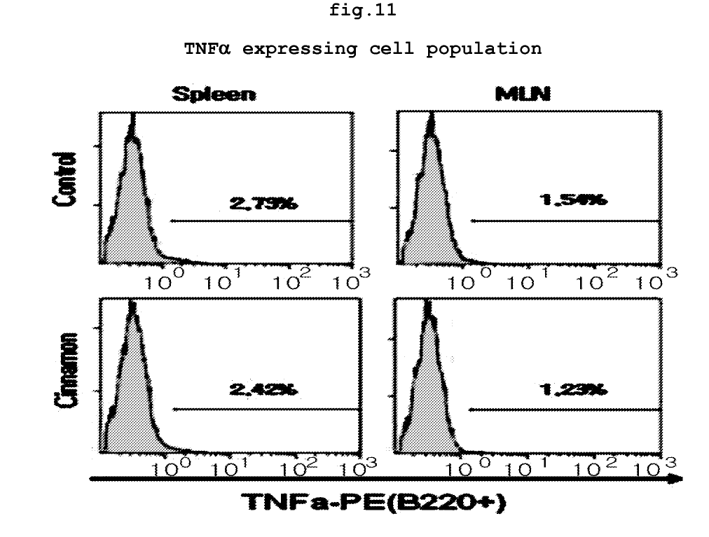

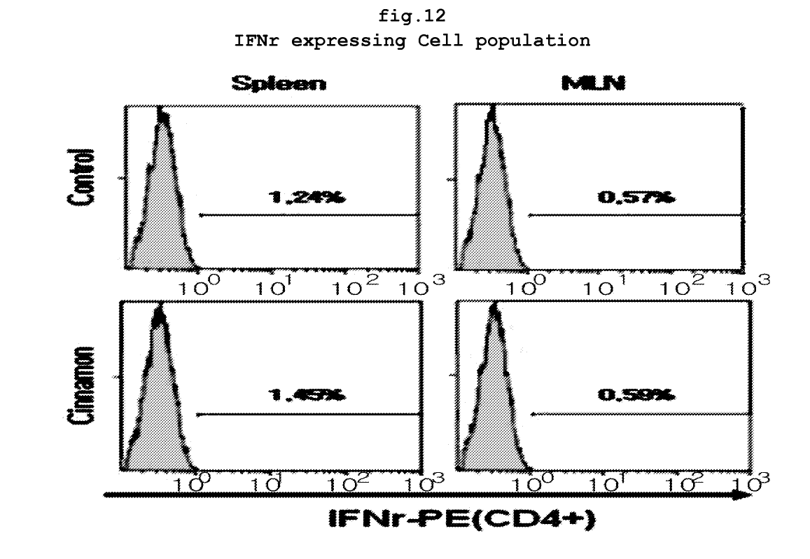

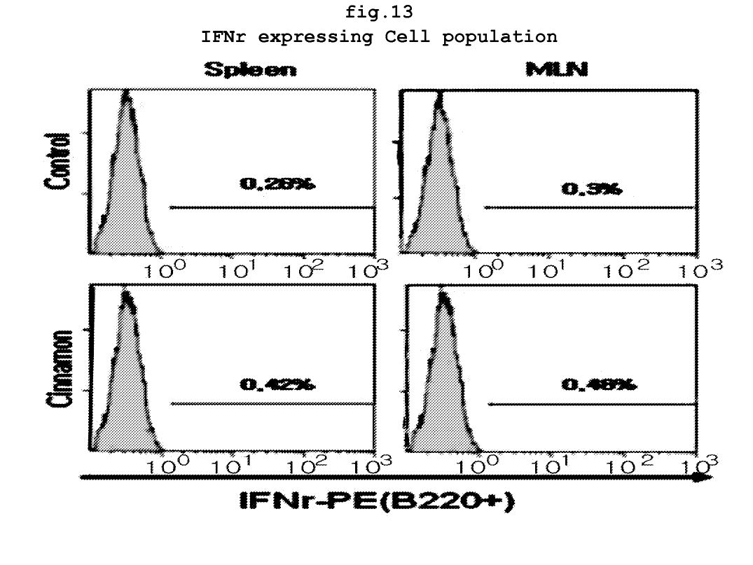

As a result, TNF-α expression was slightly increased in T cells of the mouse spleen according to the oral-administration of Cinnamomum cassia bark extract and significantly increased in T cells of the mouse mesenteric lymph node. However, INF-γ expression was not increased. B cells were also measured and there was no apparent difference between the control and the experimental group (FIGS. 10-13).

### Example 6

### The Immune Enhancing Activity of Cinnamomum Cassia Bark Extract in In Vitro Reporter Assay System

### <6-1> In Vitro Screening for Evaluating the Effect of Cinnamomum Cassia Bark Extract on the Cytokine Expression

An indirect approach was made to investigate the effect of Cinnamomum cassia bark extract on the host immune system in advance of in vivo experiment, precisely a screening for the cytokine expression was performed by using luciferase reporter assay system (TaKaRa, Japan). Promoter regions of IL2, IL4, IL10, IL24 and TNF-α were inserted into the upstream of the vector containing luciferase gene (pXPG vector, Plasmid 44, 173-182, 2000) by cloning to construct a recombinant vector in which the activity of luciferase would be regulated by the expression of cytokine promoters positioned in the upstream.

Particularly, the upstream of a vector where luciferase was expressed was digested with XhoI and KpnI. To prepare an insert, T cells were separated from a mouse and genomic DNA was extracted therefrom. The promoter region was amplified by PCR using a promoter specific primer which had XhoI and KpnI restriction enzyme sites at both ends and a starting codon at the upstream. The amplified promoter was digested with the two restriction enzymes above and purified. The prepared insert and the vector were ligated by using a ligase (New England Biolabs).

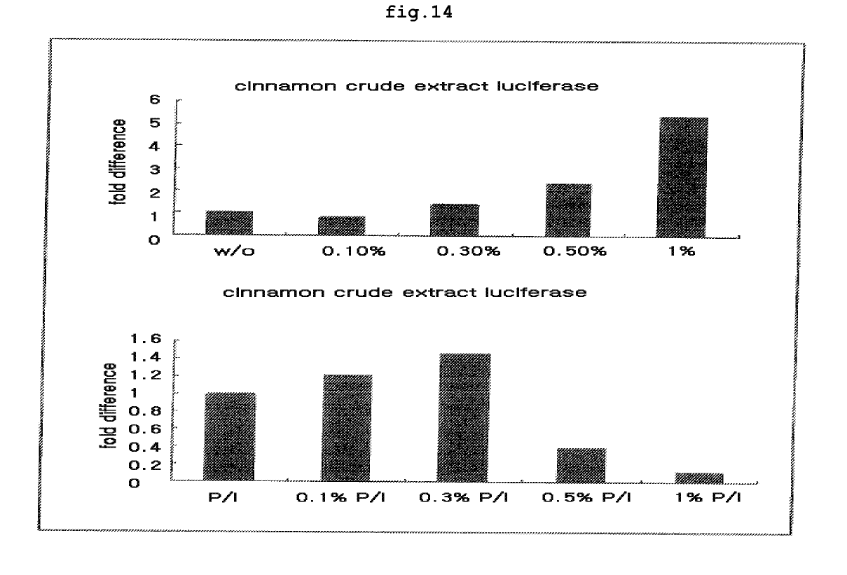

EL4 cells (ATCC; TIB-39), a kind of mouse T cell line, were transfected with the above recombinant vector by lipoplex (Invitrogen; Lipofectamine 2000). The cells were stimulated with Cinnamomum cassia bark extract only or together with PMA+ionomycin for a while. The luciferase activity was measured by using a luciferase assay kit (Dual luciferase assay system, Promega, USA). The promoters used for this experiment were the promoters of IL2, IL4, IL10, IL24 and TNF-α, but repeated experiments confirmed that only the activity of TNF-α was increased by the stimulation of Cinnamomum cassia bark extract. That is, when cells were stimulated by Cinnamomum cassia bark extract, the cytokine activity was increased Cinnamomum cassia bark extract-dose dependently. Particularly, when 1% of Cinnamomum cassia bark extract was administered, the luciferase activity was approximately 5-6 fold increased. When Cinnamomum cassia bark extract was administered together with PMA+ionomycin at the concentration of 0.1% and 0.3%, the activity was higher than that of when only PMA+ionomycin were administered (FIG. 14).

### Example 7

### Acute Toxicity in Rats Tested Via Oral Administration

6-week old SPF (specific pathogen-free) SD line rats were used in the tests for acute toxicity. The Cinnamomum cassia bark extract prepared in Example 1 was suspended in 0.5% methylcellulose solution and orally administered once to 5 rats per group at the dosage of 1 g/kg, 5 g/kg and 10 g/kg. Death, clinical symptoms, and weight changes in rats were observed, hematological tests and biochemical tests of blood were performed, and any abnormal signs in the gastrointestinal organs of chest and abdomen were checked with the naked eyes during autopsy.

The results showed that the test compounds did not cause any specific clinical symptoms, weight change, or death in rats. No change was observed in hematological tests, biochemical tests of blood, and autopsy. Therefore, the Cinnamomum cassia bark extract of the present invention is evaluated to be safe substance since it does not cause any toxic change in rats up to the level of 10 g/kg and its estimated LD₅₀ value is much greater than 10 g/kg in rats.

### Manufacturing Example 1

### Preparation of Soft Capsules

Soft capsules containing 100.0 mg of the Cinnamomum cassia bark extract prepared in Example 1, 175.0 mg of soybean oil, 45.0 mg of yellow beeswax, 127.5 mg of palm hardened oil, 21.0 mg of soybean phospholipid, 212.0 mg of gelatin, 50.0 mg of glycerin (specific gravity 1.24), 76.0 mg of D-sorbitol, 0.54 mg of paraoxymethylbenzoic acid, 0.90 mg of paraoxypropylbenzoic acid, 0.56 mg of methylvanillin and a proper amount of yellow food color 203 per capsule were prepared according to the manufacturing process of soft capsule of the Korean Pharmacopoeia.

### Manufacturing Example 2

### Preparation of Tablets

100.0 mg of the Cinnamomum cassia bark extract prepared in Example 1, 90.0 mg of cornstarch, 175 mg of lactose, 15.0 mg of L-hydroxypropylcellulose, 5.0 mg of polyvinylpyrolidone 90 and a proper amount of ethanol were mixed homogeneously and granulized by wet-granulizing method. After adding 1.8 mg of magnesium stearin thereto, tablets were prepared containing 400 mg of the mixture per tablet.

### Manufacturing Example 3

### Preparation of Capsules

100.0 mg of the Cinnamomum cassia bark extract prepared in Example 1, 83.2 mg of cornstarch, 175.0 mg of lactose and 1.8 mg of magnesium stearin were mixed homogeneously. And then, capsules were prepared containing 360 mg of the mixture per capsule.

### Manufacturing Example 4

### Preparation of Powders

| | |
|---|---|
| Cinnamomum cassia bark extract | 2 g |
| Lactose | 1 g |

Powders were prepared by mixing the above components and filling in an air tight pack.

### Manufacturing Example 5

### Preparation of Chewing Gum

Chewing gum was prepared to have the composition as follows; the Cinnamomum cassia bark extract 0.24-0.64%, gum base 20%, sugar 76.36-76.76%, fruit flavor 1%, water 2%.

### Manufacturing Example 6

### Preparation of Beverage

Beverage was prepared to have the composition as follows; the Cinnamomum cassia bark extract 0.48-1.28 mg, honey 522 mg, thioctic acid amide 5 mg, nicotinic acid amide 10 mg, sodium riboflavin hydrochloric acid 3 mg, pyridoxine hydrochloride 2 mg, inositol 30 mg, ortho acid 50 mg, water 200 ml.

### Manufacturing Example 6

### Preparation of Sausage

Sausage was prepared to have the composition as follows; the Cinnamomum cassia bark extract 0.24-0.64%, pork 63.6%, chicken meat 27.5%, starch 3.5%, soybean protein 1.7%, salt 1.62%, glucose 0.5%, other additives (glycerin) 0.94-1.34%.

### INDUSTRIAL APPLICABILITY

As explained hereinbefore, the composition containing the Cinnamomum cassia bark extract for improving intestinal flora and enhancing immune response exhibits the growth enhancing activity to Bifidobacterium longum, Lacotobacillus sp. and Lactobacillus acidophilus and also to immune cells, particularly lymphocytes of general immune system and intestinal immune system. Therefore, the composition of the invention can be developed as a therapeutic agent for intestine-related diseases such as constipation and coprostasis, an immune enhancer and health food, for example yoghurt or other dairy products, cinnamon beverages for improving digestion of aged people or patients, herb digestant, etc.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for improving intestinal flora in a subject comprising administering an effective amount of the extract of Cinnamomum cassia bark to the subject in need thereof.

2. The method according to claim 1, wherein the extract of Cinnamomum cassia bark is prepared by extracting with a solvent selected from a group consisting of water, single or mixed ether, ethanol, methanol and ethyl acetate.

3. The method according to claim 1, wherein the intestinal flora contains one or more strains selected from a group consisting of Bifidobacterium longum, Lactobacillus sp. and Lactobacillus acidophilus.

4. A method for enhancing immune response in a subject comprising administering an effective amount of the extract of Cinnamomum cassia bark to the subject in need thereof.

5. The method according to claim 4, wherein the extract of Cinnamomum cassia bark is prepared by extracting with a solvent selected from a group consisting of water, single or mixed ether, ethanol, methanol and ethyl acetate.

6. The method according to claim 4, wherein the immune enhancing effect indicates the activity of inducing immune cell proliferation.

7. The method according to claim 6, wherein the immune cells are B-lymphocytes and T-lymphocytes.

8. A method for treating constipation or intestine-related diseases in a subject comprising administering an effective amount of the extract of Cinnamomum cassia bark to the subject in need thereof.

9. The method according to claim 8, wherein the extract of Cinnamomum cassia bark is prepared by extracting with a solvent selected from a group consisting of water, single or mixed ether, ethanol, methanol and ethyl acetate.
